(19) United States
(12) Patent Application Publication
(10) Pub. No.: US 2015/0328436 A1
(43) Pub. Date: Nov. 19, 2015

(54) External Catheter Rubber Band Rings

(71) Applicant: Haim Ahrony, Encino, CA (US)

(72) Inventor: Haim Ahrony, Encino, CA (US)

(21) Appl. No.: 14/278,976

(22) Filed: May 15, 2014

(51) Int. Cl. A61M 25/02

(52) U.S. Cl. CPC A61M 25/02; A61M 2025/0213; A61M 2202/0496; A61M 2202/0014

(57) ABSTRACT

The “External Catheter Rubber Band Rings” affix over any existing sheath male external urinary catheter brand that uses a standard condom catheter and placed around the penis neck.

The External Catheter Rubber Band Rings expand and retract with the movement of the penis. An applicator for the “External Catheter Rubber Band Rings” includes a pair of Rubber Band Rings with different width sizes which affix over each other for putting on the wearer.

### BACKGROUND OF THE INVENTION

This invention is related to a male external urinary catheter and to those men who suffer from urinary incontinence that use a standard external condom catheter. The causes of men who suffer from urinary incontinence are many and diverse. Some of the reasons of urinary incontinence include but are not limited to: weak bladder muscles control due to trauma, certain prostate conditions, and nerve damage associated with surgery.

This invention is particularly designed to improve and seal the condom catheter in order to prevent it from falling off the penis.

Millions of men using the external urinary catheter have a common fear that the condom catheter would fall off which causes urine leakage. This fear prevents men from living a normal life.

Currently, there are products that use an adhesive, rubber band that rolls over the sheath, or brands that use glue with the attempt to keep the condom catheter in place. Unfortunately, due to changing body temperatures, the adhesive dissolves thereby causing the condom to fall off. Most importantly, these types of adhesives can damage the skin. The “External Catheter Rubber Band Rings” is simple, comfortable to wear, remains functional and substantially leak proof. It is the solution for men who are seeking a satisfactory solution to the problem and wish to lead an active normal life!

### SUMMARY OF THE INVENTION

The “Rubber Band Rings” affix over of any existing brand condom catheter size; and consists of two types of width sizes that will be utilized: 1) the 4 to 8 mm width and 2) the 2 to 4 mm width. The first ring is the same as any existing brand male Condom catheter in diameter size, the second ring ,is one size diameter smaller than the first ring.

First, the condom catheter must be placed over the penis. Subsequently, the first ring (4 to 8 mm width) is placed around the head of the penis. Lastly, the second ring (2 to 4 mm width) is placed over the first ring. This forms a seal over the head of the penis. The “Rubber Band Rings” are flexible and stretchy for comfort; they are durable and will ensure that catheter is secure to avoid urine leakage.

These rings are not limited to specific condom catheter brands, but can be used by all condom catheter manufactures.

### BRIEF DESCRIPTION OF THE DRAWINGS

FIG. 1:
FIG. 1.1 is a view of the first ring (4 to 8 mm width)“Rubber Band Ring” affixed over the external condom catheter around the male penis neck
FIG. 1.2 is a view of the second ring (2 to 4 mm width) “Rubber Band Ring” affixed over the first ring.
FIG. 1.3 is a view of the male penis
FIG. 1.4 is a view of the male external condom catheter showing it affixed over the penis

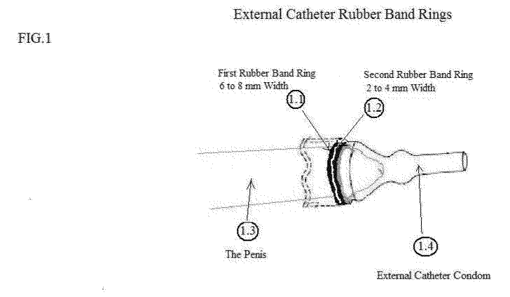

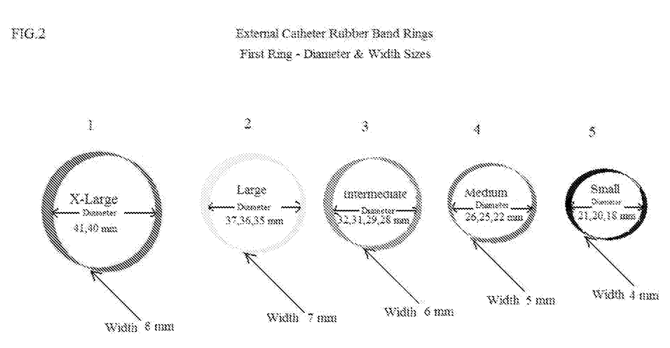

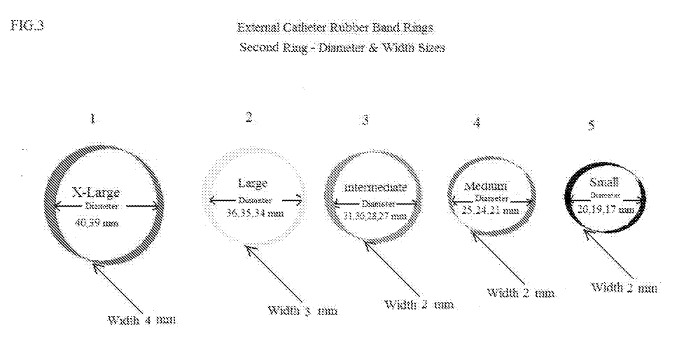

FIG. 2: is a view of the first ring (4 to 8 mm width) “External Rubber Band Rings” of diameter sizes:
X-Large: 41, 40 mm
Large: 37, 36, 35 mm
Medium: 32,31,29,28 mm
Intermediate: 32, 31, 29, 28 mm
Small: 21, 20, 18 mm

FIG. 3: is a view of the second ring (2 to 4 mm width) “External Rubber Band Rings” of diameter sizes:
X-Large: 40, 39 mm
Large: 36, 35, 34 mm
Medium: 31,30,28,27 mm
Intermediate: 31, 30, 28, 27 mm
Small: 20, 19, 17 mm

I claim:

1. External Catheter Rubber Band Rings for a wearer population, comprising:
a plurality of External Catheter Rubber Band Rings in various sizes for the wearer population, each of said External Catheter Rubber Band Rings including:
a pair of Rubber Band Rings with different width sizes which affix over the sheath male external urinary catheter and affix over each other for putting on the wearer.

2. External Catheter Rubber Band Rings as claimed in claim 1, wherein said first External Catheter Rubber Band Ring portion is of a width size bigger than a width of said second External Catheter Rubber Band Ring portion.

3. External Catheter Rubber Band Rings as claimed in claim 1 for a wearer population, comprising:
a plurality of External Catheter Rubber Band Rings in various diameters and width sizes to accommodated any existing male external urinary catheter brand.

4. External Catheter Rubber Band Rings as claimed in claim 1, said first and second External Catheter Rubber Band Rings portions being of thin elastic material affixed over the sheath for wearing on a human penis of a wearer.

5. External Catheter Rubber Band Rings as claimed in claim 1, population said first and second External Catheter Rubber Band Rings portion being selected to be of a diameter and width in a range of sizes to fit the full range of human penis sizes and full range of any existing male external urinary catheter brand.

6. External Catheter Rubber Band Rings as claimed in claim 1, the smaller width is 1 diameter in size smaller than the larger diameter size of a wearer.
